(19) United States
(12) Patent Application Publication
(10) Pub. No.: US 2022/0080144 A1
(43) Pub. Date: Mar. 17, 2022

(54) MASK BEAD WITH TUBE FEEDTHROUGH

(71) Applicant: Loewenstein Medical Technology S.A., Luxembourg (LU)

(72) Inventor: Martin EIFLER, Glueckstadt (DE)

(21) Appl. No.: 17/447,323

(22) Filed: Sep. 10, 2021

(30) Foreign Application Priority Data
Sep. 11, 2020 (DE) 102020123713.8

(51) Int. Cl. A61M 16/06

(52) U.S. Cl. CPC A61M 16/0616

(57) ABSTRACT

A mask bead for a patient interface, having at least one lip, wherein the at least one lip extends from a transition region toward a central opening and borders the central opening. At least one lip comprises at least one portion which is capable of guiding a tube between the mask bead and the facial skin of a user.

### CROSS-REFERENCE TO RELATED APPLICATIONS

The present application claims priority under 35 U.S.C. § 119 of German Patent Application No. 102020123713.8, filed Sep. 11, 2020, the entire disclosure of which is expressly incorporated by reference herein.

### BACKGROUND OF THE INVENTION

### 1. Field of the Invention

The invention relates to a mask bead for a patient interface for optional use with a tube between the mask bead and the face of a patient.

### 2. Discussion of Background Information

In some cases, patients who are being ventilated or whose breathing has to be assisted, additionally require a stomach probe to ensure a supply of nutrients, liquid or medicaments. The stomach probe is generally applied through the mouth or the nose by means of a tube. In conventional breathing masks, a high degree of leakage occurs when a tube is guided between the mask and the patient's face. In general, this leakage can be only slightly reduced by fastening the mask more tightly on the patient's face, but at the same time this causes pressure sores to develop and can make the mask uncomfortable to wear.

Various approaches have been taken in the prior art to make a breathing mask easier to wear during simultaneous use of a stomach probe. One approach uses tube feedthroughs in the mask body. Although this solution provides good sealing of the mask, it is however not possible to change or remove either the mask or the tube without having to remove the other one for quite a long period of time. Another solution proposes a separate cushion for the tube, which cushion is placed between the mask and the patient's face. A cushion of this kind can quickly feel like a foreign body and also prevents an optimal, comfortable fit of the mask. Cutouts with lamellas inside the cutout in the side regions of the mask cushion permit improved sealing during use of a tube, but, after removal of the tube, these cutouts constitute an additional loss of leak-tightness.

In view of the foregoing, it would be advantageous to have available a mask bead for a patient interface, which mask bead makes it easy to change or remove the patient interface or the tube and also provides optimal sealing and is comfortable to wear.

### SUMMARY OF THE INVENTION

The present invention provides a mask bead for a patient interface, having at least one lip, wherein the at least one lip extends from a transition region toward a central opening, wherein the at least one lip borders the central opening, at least one lip comprising at least one portion which serves for guiding a tube between the mask bead and the facial skin of a user.

In some embodiments of the mask bead, the transition region is or comprises a mask body connector.

In some embodiments of the mask bead, the material of the lip in the region of the portion is deformable and at least partially adapts to the tube.

In some embodiments of the mask bead, the portion is a cutout, which at least in some places interrupts the surface of the lip.

In some embodiments of the mask bead, the lip, without use of a tube between the mask bead and the face of the patient, bears sealingly on the face of the patient.

In some embodiments of the mask bead, the lip is configured as combined sealing lip and supporting lip.

In some embodiments of the mask bead, the sides of the cutout, without mechanical loading, have a distance with a width (B).

In some embodiments of the mask bead, the mask bead comprises at least two lips, wherein at least one lip is configured as sealing lip and at least one lip is configured as supporting lip.

In some embodiments of the mask bead, the sealing lip is designed to bear sealingly on the face of a patient.

In some embodiments of the mask bead, the supporting lip comprises the at least one portion.

In some embodiments of the mask bead, at least two lips are arranged adjacent and extend at least in part parallel to each other.

In some embodiments of the mask bead, the sealing lip is arranged on the outside and has a substantially smaller material thickness than the supporting lip, wherein the supporting lip is arranged on the inside.

In some embodiments of the mask bead, the supporting lip lies on the outside and has a substantially greater material thickness than the sealing lip.

In some embodiments of the mask bead, the sealing lip has a portion which is designed as a thin location and which has a smaller material thickness than the region of the sealing lip that surrounds the portion.

In some embodiments of the mask bead, the supporting lip comprises a portion designed as a cutout.

In some embodiments of the mask bead, the portion comprises at least one end in the direction of the central opening and also an end in the direction of the transition region.

In some embodiments of the mask bead, the end of the portion is rounded. In some embodiments of the mask bead, the end of the portion is semicircular in shape.

In some embodiments of the mask bead, the cutout is open toward the central opening.

In some embodiments of the mask bead, the end points of that end of the cutout directed toward the opening are spaced apart from each other.

In some embodiments of the mask bead, the sides of the cutout do not touch each other in the region of the end.

In some embodiments of the mask bead, the portion is configured to lie over a tube and to adapt to the tube shape at least in some regions.

In some embodiments of the mask bead, the supporting lip has a nose bridge region, two side regions and a base region, wherein the supporting lip extends from the side regions in the direction of the central opening and, in the region of the nose bridge region and of the base region, is not connected to itself.

In some embodiments of the mask bead, the supporting lip extends from the side regions and the nose bridge region in the direction of the central opening and, in the region of the base region, is not connected to itself.

In some embodiments of the mask bead, the supporting lip extends from the side regions and the base region in the direction of the central opening and, in the region of the nose bridge region, is not connected to itself.

In some embodiments of the mask bead, the lip comprises at least two portions.

In some embodiments of the mask bead, the lip comprises at least two portions, wherein at least one of the at least two portions is at least partially arranged in one of the regions of the lip extending from the side regions in the direction of the central opening.

In some embodiments of the mask bead, the lip has at least two portions which lie opposite each other, in relation to a plane (S) through the nose bridge region and the base region.

In some embodiments of the mask bead, the at least two portions have the same dimensions and geometries.

It will be noted that the features individually presented in the claims can be combined with one another in any desired, technically meaningful way and show further refinements of the invention. The description additionally characterizes and specifies the invention in particular in conjunction with the drawings.

It will also be noted that an “and/or” conjunction used herein between two features, and linking them to each other, is always to be interpreted as meaning that in a first embodiment of the subject matter according to the invention only the first feature may be present, in a second embodiment only the second feature may be present, and in a third embodiment both the first and the second feature may be present.

A ventilator is to be understood as any appliance which supports the natural breathing of a user or patient, which takes over the ventilation of the user or patient and/or which serves for respiration therapy and/or influences the respiration of the user or patient in some other way.

This includes by way of example, but not exclusively, CPAP and BiPAP appliances, anesthesia appliances, respiration therapy appliances, ventilators (for use in hospitals, in non-hospital environments or in emergencies), high-flow therapy appliances and coughing machines. Ventilators can also be understood as diagnostic appliances for respiration. Diagnostic appliances can generally be used to detect medical parameters of a patient. These also include appliances that are able to detect and optionally process medical parameters of patients in combination with respiration.

Unless specifically stated otherwise, a patient interface can be understood as any part or connected peripheral of the ventilator conceived for interaction with a patient, in particular for therapeutic or diagnostic purposes. In particular, a patient interface can be understood as a mask of a ventilator or as a mask connected to the ventilator. This mask can be a full-face mask, i.e., enclosing the nose and mouth, a total face mask (enclosing at least nose, mouth and eyes), a breathing hood or a nose mask, i.e. a mask enclosing only the nose. Tracheal tubes and so-called nasal cannulas can also be used as mask.

### BRIEF DESCRIPTION OF THE DRAWINGS

The invention is explained in more detail below on the basis of illustrative embodiments with reference to FIGS. 1 to 17.

FIG. 1 shows a mask bead in a perspective front view;

FIG. 2 shows a mask bead with two lip lamellas, in a perspective rear view;

FIG. 3 shows a detail view of the cutout from FIG. 2;

FIG. 4 shows the mask bead from FIG. 2 in a rear view;

FIG. 5 shows the section A-A through the mask bead from FIG. 4;

FIG. 6 shows the detail X from FIG. 5;

FIG. 7 shows a mask bead with two lip lamellas, in a perspective rear view;

FIG. 8 shows a detail view of the portion 16 from FIG. 7;

FIG. 9 shows the mask bead from FIG. 7 in a rear view;

FIG. 10 shows the section A-A through the mask bead from FIG. 9;

FIG. 11 shows the detail X from FIG. 10;

FIG. 12 shows a mask bead with two lip lamellas, in a perspective rear view;

FIG. 13 shows a detail view of the cutout from FIG. 12;

FIG. 14 shows the mask bead from FIG. 12 in a rear view;

FIG. 15 shows the section A-A through the mask bead from FIG. 14;

FIG. 16 shows the detail X from FIG. 15; and

FIG. 17 shows a mask bead with a cutout, in a front view.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

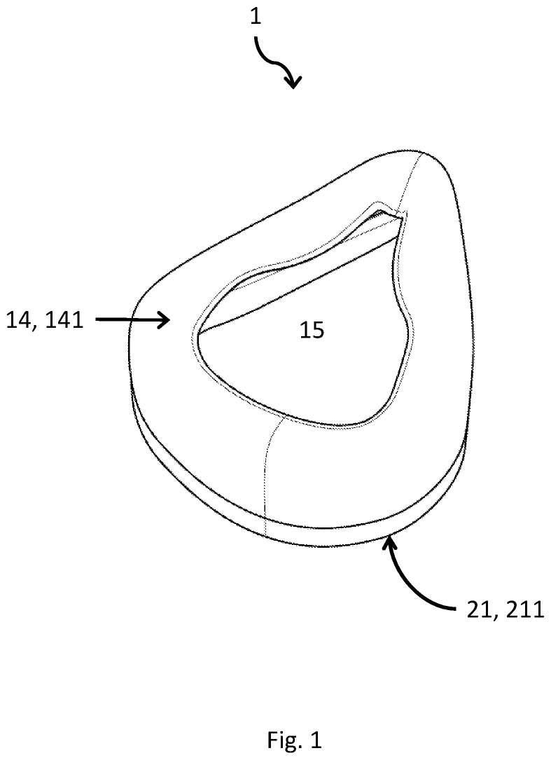

FIG. 1 shows an illustrative embodiment of the mask bead 1 in a perspective front view. The lip 14 extends in the mask bead from the transition region 21, which is designed here for example as a mask body connector 211, in the direction of a central opening 15, which is bordered completely by the lip 14. The surface 141 of the lip 14 is designed as a surface contacting the facial skin 4 of a user and terminates sealingly therewith, in particular without use of a tube 3 between mask bead 1 and facial skin 4. For example, the embodiment shown in FIG. 1 is designed to be connected to a mask body of the for example multi-part patient interface 2 and therefore has a transition region 21 designed as a mask body connector 211. In some embodiments, the patient interface 2 is also designed in one part; mask bead 1 and mask body are therefore one component, for example produced from one material. In the case of a one-part patient interface 2, the transition region 21 is a for example smooth transition between mask bead 1 and mask body. A one-part patient interface 2 can be produced for example by a two-component method and/or by injection molding. In some embodiments, the transition region 21 is designed with a greater material thickness and greater stiffness than the lip 14. The lip 14 can be designed, for example, as a combined supporting lip 143 and sealing lip 142. The two functions of supporting (supporting lip 143) and sealing (sealing lip 142) can also be divided up between two or more lips 14.

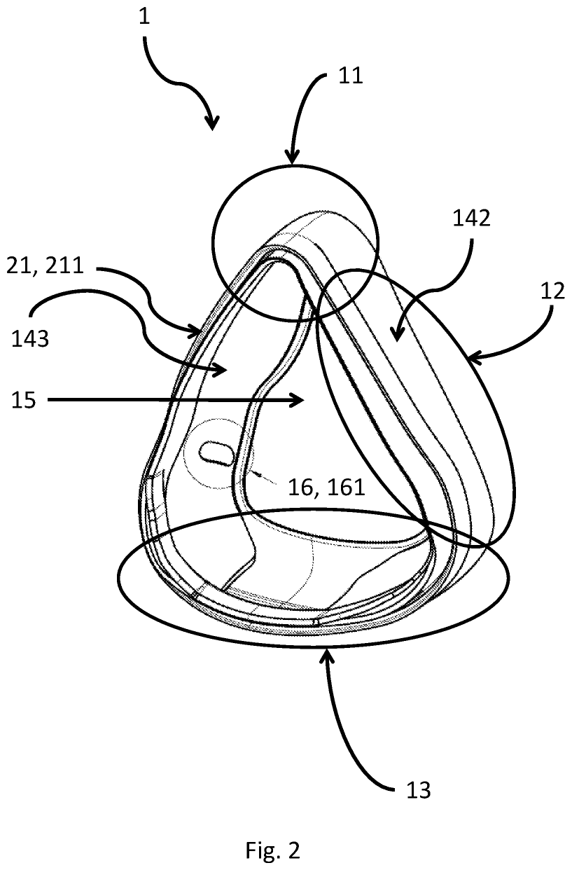

FIG. 2 shows an illustrative embodiment of the mask bead with two lips 14. The outer lip 14 is designed here as a sealing lip 142. The inner of the two lips is designed as a supporting lip 143 and additionally has a portion 16 which is designed for guiding a tube 3. The sealing lip 142 extends from the transition region 21, designed as mask body connector 211, in the direction of the central opening 15 and borders the latter completely. The supporting lip 143 also extends substantially from the transition region 21 to the central opening 15 and at least partially borders the latter.

The supporting lip 143 has for example a greater material thickness than the sealing lip 142. The strength or stiffness of the supporting lip 143 is also greater, for example as a result of the greater material thickness, in order to support the mask bead 1 or the patient interface 2 in relation to the face of the user. The sealing lip 142 has, for example, substantially a smaller material thickness than the supporting lip 143 and can adapt flexibly to the face of the user. The sealing lip 142 serves primarily to seal the mask bead 1 on the facial skin 4 of the user. In some embodiments, the material thickness of the sealing lip 142 and/or of the supporting lip 143 can also decrease and/or increase gradually from the transition region 21 in the direction of the central opening. In this way, it can happen that the sealing lip 142 has in some places a greater material thickness than the supporting lip 143. A region of the sealing lip 142 with greater material thickness (for example close to the transition region 21) can accordingly in some cases have a greater material thickness than a region of the supporting lip 143 with small material thickness (for example close to the central opening 15). Particularly in embodiments of the mask bead 1 with only one lip 14, which is designed as a combined sealing lip 142 and supporting lip 143, the lip 14 can in some places vary in its material thickness. Particularly in the region of the bearing face of the sealing lip 142 on the facial skin 4 of the user, a particularly small material thickness of the sealing lip 142 may be advantageous, which for example permits simple adaptation to the irregularities of the face of a user.

The nose bridge region 11, a side region 12 and the base region 13 are also indicated in FIG. 2. The nose bridge region 11 is the region that rests on the bridge of the user's nose. The side region 12 is to be found to the right and left of the base region 13 and nose region 11; the marking of the second side region 12 has been omitted for the sake of clarity. In the region of the base region 13 shown in FIG. 2, the side regions 12 of the supporting lip 143 are not connected or are at least not connected in some regions. Thus, in contrast to the sealing lip 142, the supporting lip does not completely border the central opening 15. The transition of the lip 14 to the transition region 21 can be clearly seen at the interruption in the base region 13 of the supporting lip 143. Particularly in the case of a patient interface 2 produced from one piece, it is possible that a smooth transition is formed at this location, for example that the material thickness gradually increases from lip 14 to transition region 21. In some embodiments, it is conceivable that the sealing lip 142 and the supporting lip 143 extend at least partially as one lip 14 from the transition region 21 in the direction of the central opening 15 and, between transition region 21 and central opening 15, divide into separate sealing lip 142 and supporting lip 143.

In some embodiments, the side regions 12 of the supporting lip 143 are also not connected in the nose bridge region 11, such that the supporting lip 143 is formed only in the side regions 12 and at most partially in the base region 13 and/or the nose bridge region 11. In some embodiments, the side regions 12 of the supporting lip 143 are also not connected only in the nose bridge region 11. In some embodiments, the side regions 12 of the supporting lip 143 are also at least partially connected in the nose bridge region 11 and/or base region 13. For example, the side regions 12 may not be connected toward the central opening 15, whereas the side regions 12 of the supporting lip 143 are connected to each other toward the transition region 21. A smaller material thickness of the supporting lip 143 in the nose bridge region 11 and/or base region 13 is also conceivable.

In a number of illustrative embodiments, the sealing lip 142 has a smaller material thickness in the nose bridge region 11 than in the other regions. The material thickness in the base region 13 of the sealing lip 142 is also smaller in some cases. The base region 13 and the nose bridge region 11 in some cases have the same material thickness, which for example is smaller than in the side regions 12 of the sealing lip 142. In a number of embodiments, the material thickness of the sealing lip 142 also gradually decreases from the side regions 12 to the base region 13 and/or nose bridge region 11. In some embodiments, the sealing lip 142 and/or the supporting lip 143, also in a combined form as an individual lip 14, can additionally have support structures.

In a side region 12 of the supporting lip 143, the mask bead 1 shown in FIG. 2 has a portion 16 which is designed and configured to guide a tube 3, which can be arranged between the mask bead 1 and the facial skin 4 of a user. If no tube 3 is arranged between the mask bead 1 and the facial skin 4 of the user, the lip 14 (primarily the sealing lip 142 in embodiments with two lips 14) seals off the mask bead 1 on the facial skin 4. If, for example, the patient interface 2 or the mask bead 1 is not worn in the correct way or the chosen size is wrong, it can also happen that the supporting lip 143, at least in some places, bears on and/or touches the facial skin 4 of the user.

The mask bead 1 with the portion 16 is therefore conceived as a mask bead for use together with a tube 3, for example for a stomach probe or the like. In addition, however, the mask bead 1 can also be used without a tube 3, without thereby having a less comfortable fit or providing less sealing compared to masks that are constructed only for use without a tube 3. The mask bead 1 shown is for example shown as a mask bead for a face mask for non-invasive ventilation. However, a number of technical features can also equally be applied to other mask types, for example to nose masks or other breathing masks.

When a tube 3 is positioned between the facial skin 4 and the sealing lip 142 of the mask bead 1 such that the tube 3 runs beneath or along the portion 16, the portion 16 settles on and/or around the tube 3. The sealing lip 142 lying between them is deformed via the tube 3 such that the sealing lip 142 runs on the tube 3, at least partially covers it and thus provides substantial sealing. Particularly in relation to a mask bead without the portion 16 conceived for the tube feedthrough, a leakage between the mask bead 1 and the facial skin of the user can be minimized when a tube 3 is used. In the illustrative embodiment in FIG. 2, the portion 16 is designed as cutout 161; in the cutout 161, the supporting lip 143 thus has no material. The surface of the supporting lip 143 is thus interrupted in the portion 16. In some embodiments, the portion 16 also be designed as a thin location, such that the portion 16 has a smaller material thickness than the surrounding regions of the lip. The width of the cutout 116 is preferably greater than the diameter of the tube 3, such that the cutout 116 can receive the tube 3. In some embodiments, a part of the sealing lip 142 arranged between the cutout 161 and the tube 3 is likewise received by the cutout 161. The cutout 161 deforms the sealing lip 142, such that the latter partially settles around the tube 3 and provides a seal at the contact points with the tube 3 and the facial skin 4. The sealing lip 142 is thus guided along the tube 3 and on the other side thereof can bear sealingly on the facial skin 4 of the user.

In some illustrative embodiments, the mask bead 1 can moreover also have several portions 16, optionally of different form and design. For example, each side region 12 of a lip 14, for example of the supporting lip 143, can have at least one portion 16. For example, each side region 12 of the supporting lip 143 can also have two or more portions 16, for example with different dimensions for different tube diameters. With portions 16 on each of the two side regions 12, a tube 3 can for example be guided from left and/or right under the mask bead 1, depending on position and design.

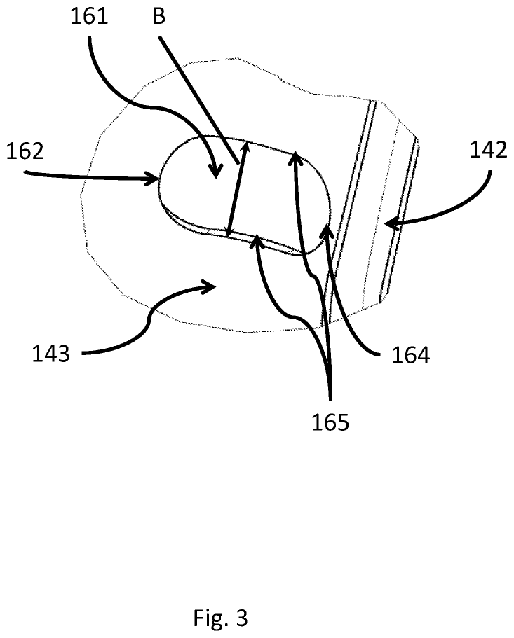

In FIG. 3, the portion 16, designed as cutout 161, of the mask bead 1 is shown enlarged in part. The portion 16 extends from an end 162, which is arranged in the direction of the transition region 21, to an end 164, which is arranged in the direction of the central opening 15. The cutout 161 is additionally bordered by the sides 165. The cutout 161 is characterized in that the cutout 161, inside the border provided by the ends 162, 164 and by the sides 165, has no material, i.e., constitutes an opening in the supporting lip 143. In the illustrative embodiment of the cutout 161 shown in FIG. 3, both ends 162 and 164 are rounded and of semicircular design. The sides 165 are substantially straight and run parallel to each other. In other illustrative embodiments, the sides 165 and/or the ends 162, 164 can also have different shapes. For example, the ends 162 and/or 164 can also be flattened or have other geometries or free forms independently of each other. In some embodiments, the sides 165 can also have a curved profile independently of each other, for example protruding is some places into the cutout 161 or having a protuberance. Depending on the length of the cutout 161, i.e., the extent from the end 162 to the end 164, it is possible that the sides 165 are not clearly distinguishable but instead merge into the shape of the end 162 and/or 164. If the cutout 161 is designed for example as a circular hole, the ends 162 and 164 can be seen as semicircles, such that no sides 165 are clearly distinguishable. If both the end 162 and the end 164 are closed, and the cutout 161 is thus not open toward the central opening 15, then the end 164 for example also settles over the tube 3.

The sides 165 have substantially a distance B from each other. This distance B is in the range of the external diameter of the tube 3, but preferably greater. In some embodiments, the distance B is also smaller than the diameter of the tube 3. In some embodiments, a plurality of portions 16 or cutouts 161 are arranged on the mask bead 1, have different distances B of the sides 165 from each other and are thus designed to receive tubes of different diameters. It is also conceivable that the distance B in some embodiments becomes smaller toward the end 162 for example. For example, a cutout 161 can also receive different tube diameters.

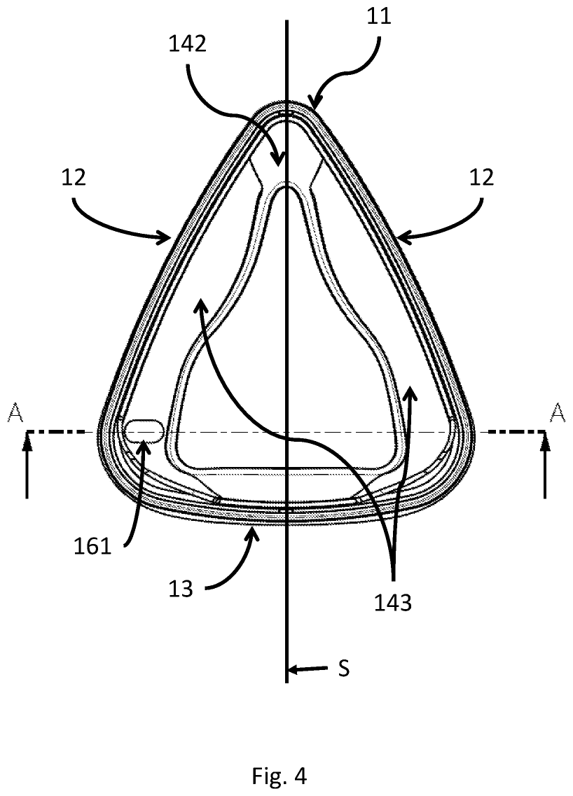

FIG. 4 shows the example of the mask bead in a rear view, i.e., looking at the transition region 21 designed as mask body connector 211. In the illustrative embodiment shown, the supporting lip 143 has a cutout 161 in the side region 12, close to the base region 13. By way of example, an embodiment is shown in which the side regions 12 of the supporting lip 143 are not connected to each other in the nose bridge region 11 and in the base region 13. Moreover, FIG. 4 also indicates the plane S running through the nose bridge region 11 and the base region 13. In a number of embodiments, at least two portions 16 are arranged in the mask bead 1, which two portions 16 lie opposite each other in relation to the plane S and for example have the same geometry and dimensions (length and width). For example, two or more portions 16, for example of different geometry and dimensions, can also be arranged in one side region 12, which portions 16 are likewise arranged, correspondingly mirrored on the plane S, in the opposite side region 12.

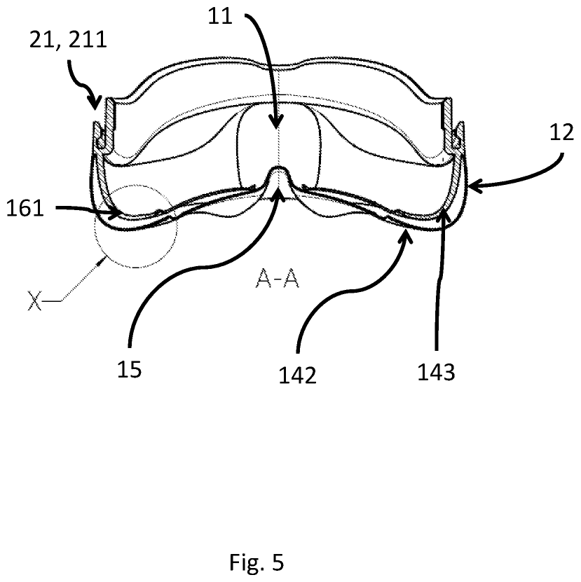

FIG. 4 also shows a section A-A through, inter alia, the cutout 161 which is shown in FIG. 5. FIG. 5 shows the in parts parallel profile of the sealing lip 142 and of the supporting lip 143 from the transition region 21 or the mask body connector 211 to the central opening 15. In the illustrative embodiment, the sealing lip 142 runs on the outside, such that it can rest on the facial skin 4 of a user. In an embodiment with two lips 14, the outer lip 14 is therefore the lip 14 that is farthest away from the geometric center of gravity of the mask bead with the greatest surface area. The material thickness of the sealing lip is substantially constantly smaller than the material thickness of the supporting lip 143. The supporting lip 143 has, for example, a material thickness gradually decreasing from the transition region 21 in the direction of the central opening 15, and therefore the supporting lip 143 can support the mask bead 1. The smaller material thickness toward the central opening permits, for example, support of the sealing lip 142 on the face of the user without exerting uncomfortable pressure. The material thickness and/or stiffness or strength of the supporting lip should however be great enough to ensure that the sealing lip 142 can be guided over the tube 3. In some embodiments, the supporting lip 143 also serves, without use of a tube 3, to allow the sealing lip 142 to bear sealingly on the facial skin 4 of the user and help against leakages, for example at high respiratory pressures. In some embodiments, the mask bead 1 is made from one material, wherein the different mechanical properties are obtained primarily by a variation in the geometry and in the material thickness. In some embodiments, however, parts of the mask bead can also be made from different materials. For example, a more elastic/more extensible material can be chosen for the sealing lip 142 compared to that for the supporting lip 143. The supporting lip 143 can by contrast be made of a somewhat stiff material. In this way, it is possible to achieve a better supporting effect and/or dimensional stability of the supporting lip 143. By means of a very extensible sealing lip 142, it is also possible for the sealing lip 142 more easily to rest sealingly around the tube 3, wherein the cutout 161 of the supporting lip 143 contributes to ensuring that the sealing lip 142 settles at least partially around the tube 3.

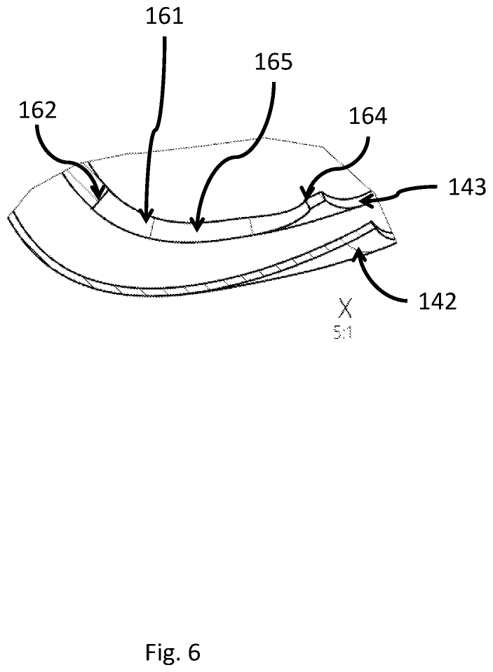

The part marked with X in FIG. 5 is shown in detail in FIG. 6. The outer sealing lip 142 has a smaller material thickness than the inner supporting lip 143. For example, an abrupt or stepped transition from the supporting lip 143 to the cutout 161 can be seen. In some embodiments, this transition can also be smooth, i.e., the material thickness decreases gradually in the direction of the center point of the cutout and finally merges into the cutout free of material.

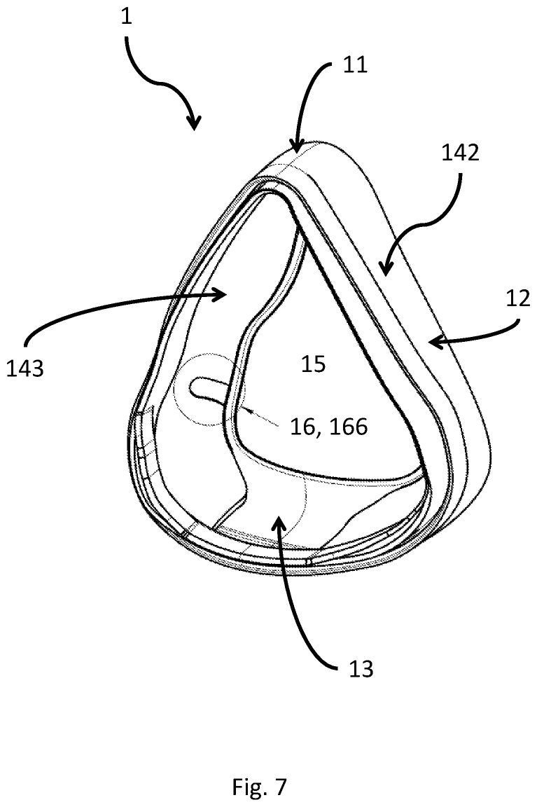

FIGS. 7 to 11 show a further illustrative embodiment of the mask bead 1. The mask bead 1 here corresponds in large part to the mask bead described with reference to FIGS. 2 to 6. In contrast to the previously described mask bead, the portion 16 here is designed as a thin location 166. In contrast to the cutout 161, the thin location 166 does not constitute an interruption of the supporting lip 143 but has a considerably smaller material thickness compared to the rest of the supporting lip 143. In some embodiments, the thin location 166 is also made of a different material, for example a more elastic material, than the supporting lip 143.

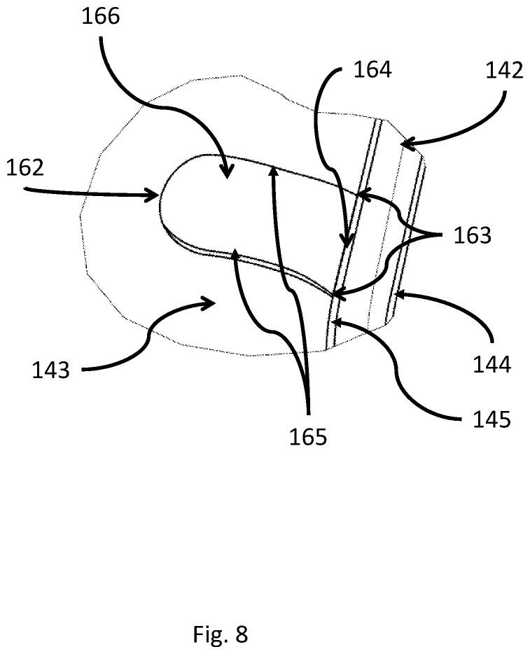

In the illustrative embodiment shown, the end 164 of the thin location 166 reaches as far as the edge 145 of the supporting lip 143 to the central opening 15, as is indicated in FIG. 8. Since the end 164 of the thin location is not rounded, the end points 163 of the thin location 166 can also be seen. It is also conceivable that the end 164 of the thin location 166 does not reach quite as far as the edge 145.

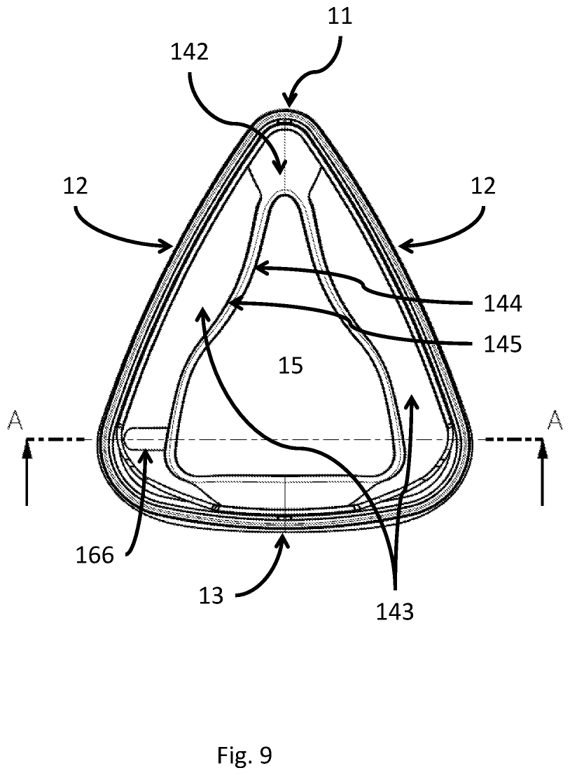

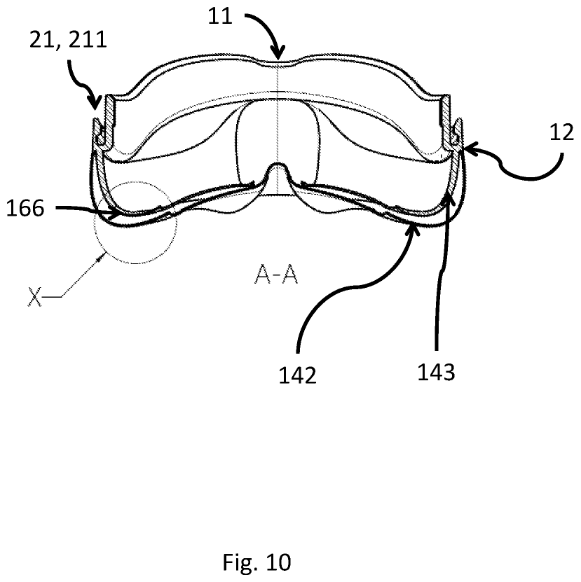

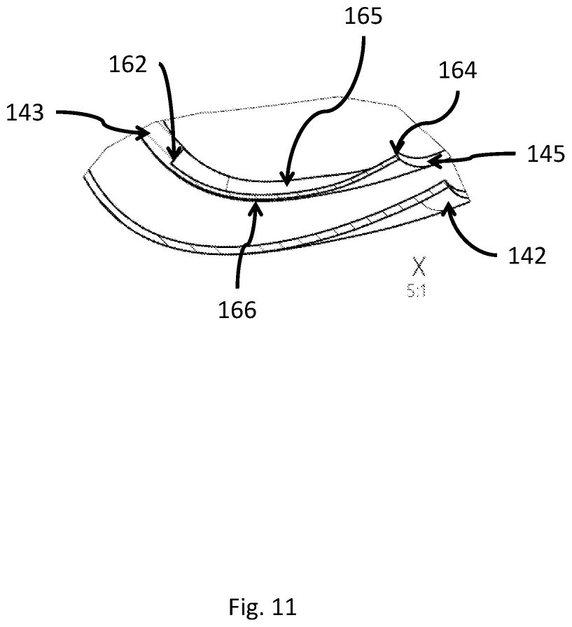

FIG. 11 shows the detail X of FIG. 10 from the section A-A (FIG. 9) through the mask bead 1. The material thickness of the supporting lip 143 changes abruptly, for example, at the transition into the thin location 166. The abrupt transition shown here runs in a stepped shape on the inner side of the supporting lip 143, i.e., on the side directed away from the user's face during use. However, the transition can also run in a stepped shape on the outer side, i.e., the side directed toward the user's face. A stepped transition on both sides of the supporting lip 143 may also be possible, for example.

In some embodiments, the transition to the thin location 166 can also be formed by a gradual decrease in the material thickness. The start of the thin location 166 can be represented, for example, by the start or the end of the gradual decrease. A definition of a transition region to the thin location 166 is also possible. The thin location 166 is generally embodied with a substantially homogeneous material thickness, but in some embodiments the material thickness can also vary within the thin location 166.

In relation to the rest of the supporting lip 143, the thin location 166 has a much lesser stiffness and/or strength, e.g., contiguous with the smaller material thickness or also through choice of another material, and is more extensible compared to the rest of the supporting lip 143. This lesser stiffness and/or greater extensibility permits that the supporting lip 143 can place or guide both the thin location 166 and the sealing lip 142 at least partially around the tube 3. As a result of the stiffer and/or thicker side regions at the sides 165 and the end 162 of the supporting lip 143, the sealing lip 142 is deformed such that a part of the sealing lip 142 fits on the tube and is pressed or pulled at the sides of the tube 3 as far as the facial skin 4 of the user and bears thereon.

If a plurality of portions 16 are arranged on the mask bead 1, these can be designed partially as cutouts 161 and partially also as thin locations 166. In some embodiments, it is possible that an additional thin location 166, for example, is arranged in the sealing lip 142 below or parallel to a cutout 161 in the supporting lip 143. In some embodiments, the sealing lip 142 can have a cutout 161, and the supporting lip 143 can have a thin location 166. Moreover, it is also conceivable, for example, that sealing lip 142 and supporting lip 143 have superposed thin locations 166 and/or cutouts 161. It is also conceivable that the superposed portions have different geometries and/or sizes. For example, the portion 16 of the sealing lip 142 can be wider and/or longer (i.e., distance between the ends 162 and 164) than the portion 16 of the supporting lip 143. It may also be possible that the portion 16 of the supporting lip 143 is wider and/or longer than the portion 16 of the sealing lip 142.

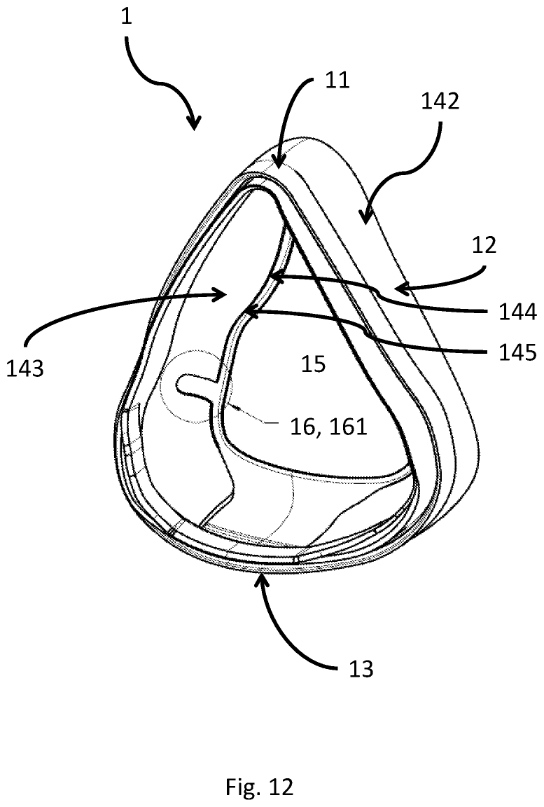

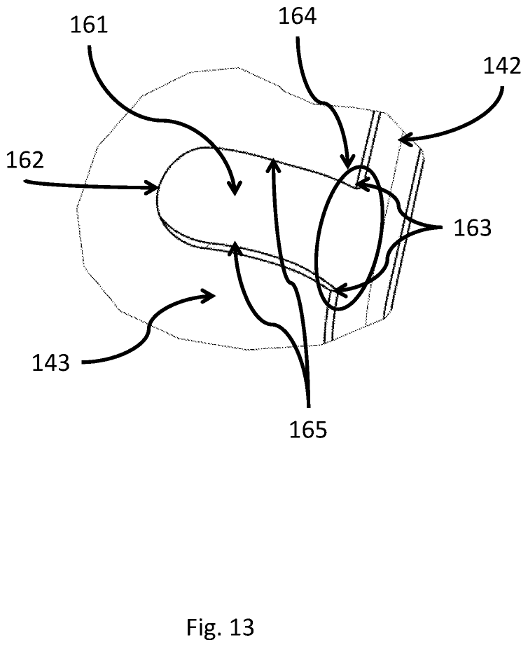

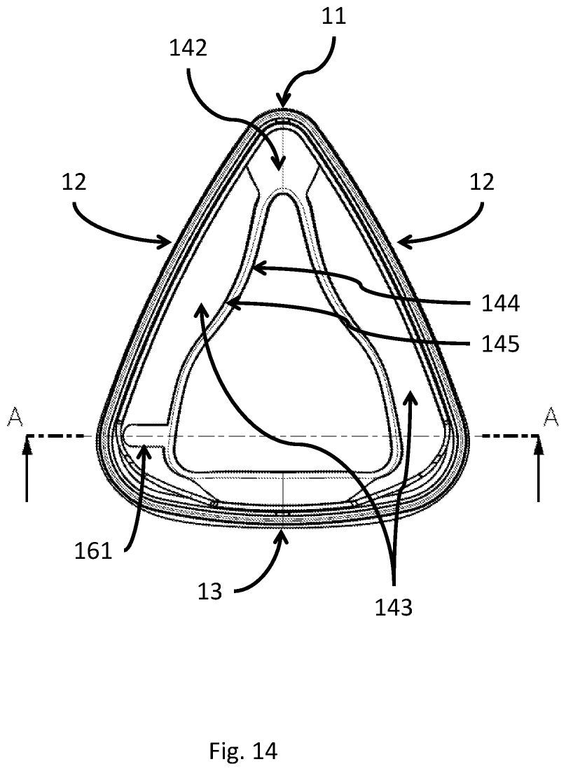

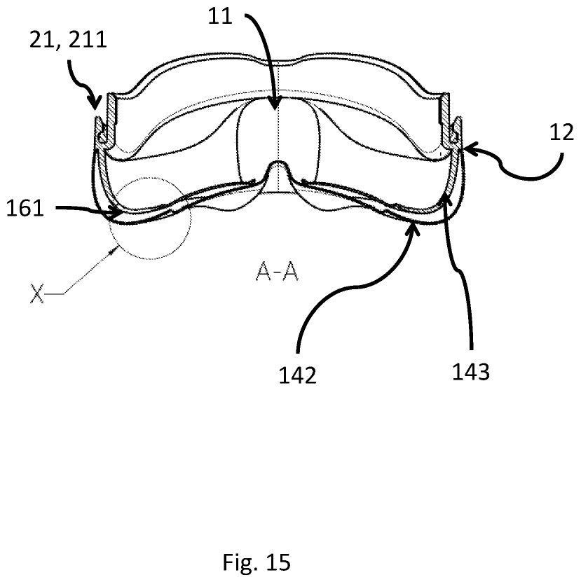

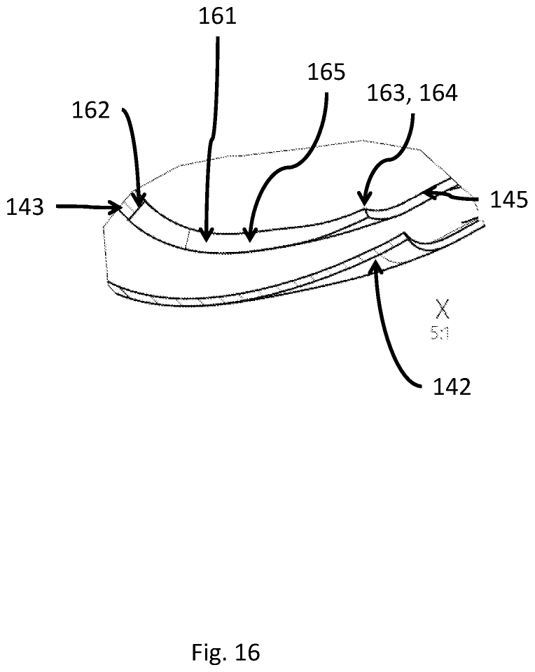

FIGS. 12 to 16 show schematic views of a further illustrative embodiment of the mask bead 1. The embodiment shown in FIGS. 12 to 16 differs from the previously described embodiments in that the portion 16 is a cutout 161 which is open toward the central opening 15. The end 164 of the cutout 16 thus reaches as far as the edge 145 of the supporting lip 143, wherein the end points 163 of the cutout 161 are not directly connected to each other and do not touch each other at least when no mechanical load, for example caused by placing the mask bead 1 on the face of a user and tightening the straps, is acting on the mask bead 1.

When a tube 3 is used between the mask bead 1 and the facial skin 4 of the user, the sealing lip 142 is at least partially pushed or pressed through the cutout 161 over the tube 3. The sealing lip 142 can deform over the tube 3 into the portion 16, here as cutout 161, and thus at least partially fit around the tube 3.

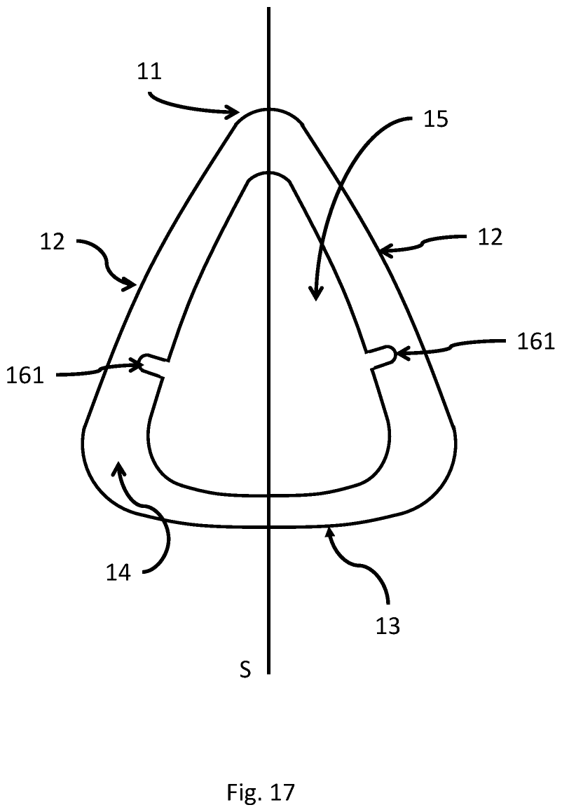

FIG. 17 shows an illustrative embodiment of the mask bead 1 in a front view. The mask bead 1 has a lip 14 which is designed as combined sealing lip 142 and supporting lip 143. In the embodiment shown, the lip 14 has, in each of the two side regions 12, a portion 16 designed as a cutout 161. The two cutouts 161 lie opposite each other in relation to the plane S through the base region 13 and the nose bridge region 11. The portions 161 are open toward the central opening 15 and thus form an interruption of the surface of the lip 14. When a tube 3 is used together with the mask bead 1, the cutout 161 can receive the tube 3, such that the sides 165 of the cutout 161 reach along the sides of the tube as far as the facial skin 4 of the user. In some embodiments, the end points 163 of the cutout 161 can be shaped such that they run toward each other, but do not touch each other, without mechanical loading (on the mask bead). If the end points 163 are shaped such that they form a partial circle, for example, they can settle on the facial skin 4 under the tube 3 and thereby further reduce any leakage of the mask bead 1.

To sum up, the present invention provides the following items:
1. A mask bead for a patient interface, wherein the mask bead comprises at least one lip, the at least one lip extending from a transition region toward a central opening and bordering the central opening, and wherein at least one lip comprises at least one portion which is capable of guiding a tube between the mask bead and a facial skin of a user.
2. The mask bead of item 1, wherein the transition region is or comprises a mask body connector.
3. The mask bead of item 1 or item 2, wherein the material of the at least one lip in a region of the at least one portion is deformable and at least partially adapts to the tube, and wherein the at least one lip, without the use of a tube between the mask bead and the face of a patient, bears sealingly on the face of the patient.
4. The mask bead of any one of the preceding items, wherein the at least one portion is a cutout, which at least in some places interrupts the surface of the at least one lip.
5. The mask bead of any one of the preceding items, wherein the at least one lip is configured as combined sealing lip and supporting lip.
6. The mask bead of any one of items 1 to 4, wherein the mask bead comprises at least two lips, at least one lip being configured as sealing lip and at least one lip being configured as supporting lip.
7. The mask bead of any one of items 5 and 6, wherein the supporting lip comprises the at least one portion.
8. The mask bead of any one of items 5 to 7, wherein the sealing lip is arranged on the outside and has a substantially smaller material thickness than the supporting lip, the supporting lip being arranged on the inside.
9. The mask bead of any one of items 5 to 7, wherein the supporting lip lies on the outside and has a substantially greater material thickness than the sealing lip.
10. The mask bead of any one of items 5 to 9, wherein the sealing lip comprises a portion which is designed as a thin location and has a smaller material thickness than the region of the sealing lip that surrounds the at least one portion.
11. The mask bead of any one of items 5 to 10, wherein the supporting lip comprises the at least one portion configured as cutout.
12. The mask bead of any one of the preceding items, wherein the at least one portion comprises at least one first end in the direction of the central opening and also a second end in the direction of the transition region, the second end of the at least one portion being rounded, preferably semicircular in shape.
13. The mask bead of any one of items 11 and 12, wherein the cutout is open toward the central opening, the sides of the cutout not touching in the region of the second end.
14. The mask bead of any one of items 11 to 13, wherein the end points of the end of the cutout that is directed toward the opening are spaced apart from each other.
15. The mask bead of any one of the preceding items, wherein the at least one portion is configured to lie over a tube and to adapt to a tube shape at least in some regions.
16. The mask bead of any one of items 5 to 11, wherein the supporting lip comprises a nose bridge region, two side regions and a base region and extends from the side regions in the direction of the central opening and, in a region of the nose bridge region and of the base region, is not connected to itself.
17. The mask bead of any one of items 5 to 11, wherein the supporting lip extends from the side regions and the base region in the direction of the central opening and, in the region of a nose bridge region, is not connected to itself.
18. The mask bead of any one of the preceding items, wherein the at least one lip comprises at least two portions which are capable of guiding a tube between the mask bead and the facial skin of a user.
19. The mask bead of any one of the preceding items, wherein the at least one lip comprises at least two portions which are capable of guiding a tube between the mask bead and the facial skin of a user, at least one of the at least two portions being at least partially arranged in one of at least two regions of the at least one lip which extend from the side regions in the direction of the central opening.
20. The mask bead of any one of the preceding items, wherein the at least one lip comprises at least two portions which are capable of guiding a tube between the mask bead and the facial skin of a user and are opposite each other, in relation to a plane (S) through the nose bridge region and the base region.

### LIST OF REFERENCE NUMERALS

1 mask bead
2 patient interface
3 tube
4 facial skin
11 nose bridge region
12 side region
13 base region
14 lip
15 opening
16 portion
21 transition region
141 surface
142 sealing lip
143 supporting lip
161 cutout
162 end
163 end point
164 end
165 side
166 thin location
211 mask body connector

What is claimed is:

1. A mask bead for a patient interface, wherein the mask bead comprises at least one lip, the at least one lip extending from a transition region toward a central opening and bordering the central opening, and wherein at least one lip comprises at least one portion which is capable of guiding a tube between the mask bead and a facial skin of a user.

2. The mask bead of claim 1, wherein the transition region is or comprises a mask body connector.

3. The mask bead of claim 1, wherein a material of the at least one lip in a region of the at least one portion is deformable and at least partially adapts to the tube, and wherein the at least one lip, without use of a tube between the mask bead and a face of a patient, bears sealingly on the face of the patient.

4. The mask bead of claim 1, wherein the at least one portion is a cutout, which at least in some places interrupts a surface of the at least one lip.

5. The mask bead of claim 1, wherein the at least one lip is configured as combined sealing lip and supporting lip.

6. The mask bead of claim 1, wherein the mask bead comprises at least two lips, at least one lip being configured as sealing lip and at least one lip being configured as supporting lip.

7. The mask bead of claim 6, wherein the supporting lip comprises the at least one portion.

8. The mask bead of claim 6, wherein the sealing lip is arranged on an outside and has a substantially smaller material thickness than the supporting lip, the supporting lip being arranged on an inside.

9. The mask bead of claim 6, wherein the supporting lip lies on an outside and has a substantially greater material thickness than the sealing lip.

10. The mask bead of claim 6, wherein the sealing lip comprises a portion which is designed as a thin location and has a smaller material thickness than a region of the sealing lip that surrounds the at least one portion.

11. The mask bead of claim 6, wherein the supporting lip comprises the at least one portion configured as cutout.

12. The mask bead of claim 1, wherein the at least one portion comprises at least one first end in a direction of the central opening and also a second end in a direction of the transition region, the second end of the at least one portion being rounded.

13. The mask bead of claim 11, wherein the cutout is open toward the central opening, sides of the cutout not touching in a region of the second end.

14. The mask bead of claim 11, wherein end points of the end of the cutout which is directed toward the opening are spaced apart from each other.

15. The mask bead of claim 1, wherein the at least one portion is configured to lie over a tube and to adapt to a tube shape at least in some regions.

16. The mask bead of claim 6, wherein the supporting lip comprises a nose bridge region, two side regions and a base region and extends from the side regions in a direction of the central opening and, in a region of the nose bridge region and of the base region, is not connected to itself

17. The mask bead of claim 6, wherein the supporting lip extends from side regions and a base region in a direction of the central opening and, in a region of a nose bridge region, is not connected to itself.

18. The mask bead of claim 1, wherein the at least one lip comprises at least two portions which are capable of guiding a tube between the mask bead and a facial skin of a user.

19. The mask bead of claim 1, wherein the at least one lip comprises at least two portions which are capable of guiding a tube between the mask bead and a facial skin of a user, at least one of the at least two portions being at least partially arranged in one of at least two regions of the at least one lip which extend from side regions in a direction of the central opening.

20. The mask bead of claim 1, wherein the at least one lip comprises at least two portions which are capable of guiding a tube between the mask bead and a facial skin of a user and are opposite each other in relation to a plane (S) through a nose bridge region and a base region.
